(19) United States
(12) Patent Application Publication
(10) Pub. No.: US 2005/0202067 A1
(43) Pub. Date: Sep. 15, 2005

(54) Matrix structure and hybrid matrix system for inducing a neofacia, their use and method for generating a neofacia

(75) Inventors: Andreas Lendlein, Berlin (DE); Karola Lutzow, Berlin (DE); Steffen Kelch, Berlin (DE); Thomas Groth, Berlin (DE); Heinz Johannes Buhr, Berlin (DE); Christoph Isbert, Furth (DE); Christoph-Thomas Germer, Nurnberg (DE); Christoph Reissfelder, Berlin (DE); Jorg-P. Ritz, Berlin (DE)

Correspondence Address: NORRIS, MCLAUGHLIN & MARCUS, 875 THIRD AVE, 18TH FLOOR, NEW YORK, NY, 10022, US

(73) Assignee: GKSS Forschungszentrum, Geesthacht (DE)

(21) Appl. No.: 10/974,541

(22) Filed: Oct. 27, 2004

(30) Foreign Application Priority Data
Oct. 28, 2003 (DE) DE 103 50 834.1

(51) Int. Cl. A61K 9/70

(52) U.S. Cl. 424/443; 424/93.7

(57) ABSTRACT

The invention is directed to a matrix structure for inducing a neofascia with at least one two-dimensional matrix of at least one biocompatible and resorbable polymer, wherein the surface of the matrix is designed to immobilize tissue cells, in particular autologous fibrocytes of the fascia transversalis, and to reduce a preferably three-dimensional tissue growth in vitro or in vivo. The invention is also directed to a hybrid matrix system, which includes the matrix structure and tissue cells immobilized thereon. The novel matrix as well as the fascia hybrid are characterized by a high biocompatibility and overcome the disadvantages of non-degradable implants, thereby promoting formation of a neofascia with native biomechanical properties.

The invention relates to an implantable matrix structure for inducing a neofacia; a hybrid matrix system with such matrix structure as well as tissue cells immobilized thereon; their use in hernia surgery; a method and a kit for generating a neofacia; a neofacia produced with this method, as well as a method for treatment of hernias.

The surgical therapy of hernias, in particular of hiatal wall and inguinal hernias is one of the most common surgical procedures in the Western industrialized nations. For example, approximately 200,000 hernia operations are performed yearly in Germany and more than 500,000 hernia operations in the United States. A significant problem with all conventional surgical procedures is the recurrence (relapse) of a hernia after an operation. Social economic aspects also play an important role in addition to medical issues, because a second operation may result in a prolonged disability and additional expenses.

Whereas in the past the hernia opening was closed with stitches, initially conventionally, later also by minimally invasive techniques, non-resorbable synthetic meshes have been used increasingly since the beginning of the '90s for treating hiatal and inguinal hernias (S. Anwar: The use of prosthetics in hernia repair. Hosp. Med. 2003, 64(1), 34-35), which has significantly lowered the rate of recurrence (EU Hernia Trialists Collaboration: Repair of inguinal hernia with synthetic mesh; meta-analysis of randomized controlled trials. Ann. Surg. 2002, 235 (3), 322-332). The use of completely resorbable synthetic meshes is also known. However, synthetic meshes were not successful due to increased scar tissue formation and consecutive recurrence of the hiatal and inguinal defects (H. R. Willem: Vicryl cushion in the therapy of inguinal and hiatal hernia by introduction of strong scar tissue. Chirurg. 1987, 58 (4), 300-302).

U.S. Pat. No. 5,700,583 A, U.S. Pat. No. 5,645,850 A, and the U.S. Pat. No. 5,597,579 A disclose completely resorbable, biocompatible materials for producing different medical and/or surgical components that can brake down within hours or several days. The material is not intended to be populated with cells. EP 0 841 879 B, U.S. Pat. No. 6,755,867 B, and U.S. Pat. No. 6,755,868 B disclose plug-shaped, implantable hernia prosthetics in form of corrugated funnels made of non-resorbable materials, which are inserted into the defect location. The surrounding tissue is expected to grow into the openings of the implant after implantation. However, this process does not represent a population of the material with cells. A hernia prosthetics of a non-resorbable material that is anchored in the surrounding tissue with attachment elements having a shape memory is disclosed in EP 1 083 842 B. U.S. Pat. No. 6,737,371 B describes a hernia implant made of two interwoven mesh fabrics, wherein one of the mesh fabric is made of a non-resorbable material and the other mesh fabric is made of a resorbable material.

One problem associated with the implantation of non-resorbable synthetic meshes is related to unexpected long-term side effects, prompting a warning against hasty use in hernia surgery. Tests on human explanted synthetic meshes show a significantly higher cell profilation and apoptosis around the region of the foreign material after an average implantation time of approximately two years (B. Klosterhalfen et al.: Pathology of traditional surgical mesh for hernia repair of the long-term implantation in the human. Chirurg. 2000, 71 (1), 41-42). Early animal studies have shown that the implantation of various synthetic foils can induce sarcoma (R. F. Davies: Peri-renal sarcoma induced by cellulose wrapping. Nature 1965,207 (995), 420). In addition, the synthetic meshes currently used in humane medicine have a number of additional disadvantages, for example an increased risk for infections, tissue shrinkage, and excessive scar formation (U. Klinge et al.: Do multifilament allosynthetic meshes increase the infection rate? Analysis of the polymeric surface, the bacteria adherents, and the in vivo consequences in a rat model. J. Biomol. Mater. Res. 2002, 63 (6), 765-771). In addition, the mechanical properties of current materials do not match those of natural tissue structures. In particular, the current materials are too stiff and inflexible as a result of the required mechanical load carrying capacity.

It is therefore an object of the invention to provide an implantable matrix for surgical treatment of hernias, which have an improved bio-compatibility over known materials and have mechanical properties that approach those of natural tissue structures. The material should also have the lowest possible recurrence rate after implantation. Moreover, a matrix system produced by Tissue Engineering should be provided for implantation.

The object is solved, on one hand, by an implantable matrix structure for inducing a neofacia with the features of claim 1. The matrix structure of the invention includes at least one two-dimensional matrix made of at least one biocompatible synthetic polymer that can be broken down in vivo within a predetermined resorption time, wherein the matrix has a surface capable of immobilizing tissue cells and inducing formation of tissue on the surface in vitro and/or in vivo. The embodiment according to the invention of the matrix surface provides for the initial population with tissue cells and subsequent formation of contiguous tissue structures and finally of a self-supporting neofacia that is quite similar to the natural structure to be repaired. The almost natural properties of the formed neofacia obviate the need for stabilizing a permanent implant, so that a completely resorbable material can be used instead. A non-resorbable implant with its potentially harmful properties is therefore not needed.

The object of the invention is also solved by a hybrid matrix system according to claim 21. The hybrid matrix system of the invention includes a matrix structure according to the invention with at least one two-dimensional synthetic matrix made of a biocompatible synthetic polymer and with tissue cells immobilized on the synthetic matrix. A product made in this manner by Tissue Engineering aids in a rapid regeneration of the tissue to be repaired after implantation through formation of a neofacia.

The matrix structure of the invention and the hybrid matrix system, respectively, can be used for repairing the variety of tissue tears. According to another aspect of the invention, it is preferably used for treating hernias, in particular as implant for the repair of hiatal or inguinal hernias. For this purpose, either the (unpopulated) matrix structure of the invention or the hybrid matrix system of the invention, i.e., the matrix structure that is populated more or less densely in vitro with tissue cells, are used.

The invention is also directed to a method for generating a neofacia, wherein a matrix structure according to the invention made of at least one resorbable synthetic biocompatible polymer is used and wherein a surface of the matrix is at least partially populated in vitro and/or in vivo with tissue cells and growth of the immobilized cells is induced on the matrix surface to form the neofacia.

The invention is further directed to a kit which includes the matrix structure of the invention or the hybrid matrix system of the invention as well as media for storing the structure or the system and/or media for cultivating tissue cells and/or tissues. The media can include suitable nutrients, growth factors as well as buffer systems or additional constituents.

The invention also relates to a method for treating hernias, in particular of hiatal or inguinal hernias, wherein primary or conditioned tissue cells of a patient are immobilized on a matrix structure according to one of claims 1 to 20, are optionally cultivated, and the matrix with the cells immobilized thereon is implanted.

Additional advantageous embodiments of the invention are recited in the dependent claims.

Matrix Structure

Preferably, the matrix structure of the invention has mechanical properties that substantially match those of the tissue to be repaired or of the tissue to be generated. More particularly, the required mechanical load carrying capacity of the polymeric synthetic matrix should at least approximately match that of the tissue to be generated and should also last at least until a self-supporting tissue structure is formed from the immobilized cells. The patient can then be discharged early after the surgical implantation of the matrix system. Tests on human cadavers have shown a tensile strength of the fascia transversalis at the primary suture of at least 50 N with an elasticity of more than 4 N/cm. A tensile strength of the matrix of at least 35 N, in particular 40 N, preferably at least 50 N, is preferred. The elasticity of the matrix has advantageously value of at least 2.5 N/cm, in particular at least 3.0 N/cm, and preferably at least 4.0 N/cm.

The matrix is made of a synthetic polymer that is essentially completely resorbable in vivo. Preferably, the in vivo resorption time of the polymer is selected so that a resorption-related change of the bio-mechanical matrix properties, for example of the tensile strength and the elasticity, are compensated at least approximately by the tissue structure formed on the matrix. The diminishing load carrying capacity of the synthetic matrix structure is thereby successively compensated by the increasing load carrying capacity of the tissue, so that the average bio-mechanical properties remain constant. More particularly, the resorption time should be selected so that the matrix is completely broken down only when a mechanically load-bearing tissue is already established. The preferred resorption times of the polymer, during which the matrix almost completely degrades in vivo, can reach approximately 12 months, preferably between 3 and 6 months. It is important that the implant according to the invention provides the stability and load carrying capacity required for repairing the hernia, until the cells have formed a supporting neofacia. The resorption times as well as the structure of the polymer should therefore match the time required for forming the new tissue structure.

Preferably, the synthetic matrix has the form of a membrane or a foil or has a mesh-like structure. Especially for relatively long resorption times, the membrane or foil is advantageously also provided with pores that promote an exchange of nutrients and metabolic products. The synthetic matrix can have a rough or smooth surface. It has preferably a surface texture that promotes immobilization of tissue cells, in particular of fibrocytes.

According to a particularly preferred embodiment of the invention, the surface of the synthetic matrix is structured to induce oriented three-dimensional tissue growth. For this purpose, the surface structure is preferably straight or wavy, and/or has groove-shaped or point-shaped recesses on the matrix surface. For example, the structure can be applied to the matrix during or after its fabrication. In this way, an “anisotropic” fascia tissue similar to the natural fascia can be induced along a preferred direction. The same result can be obtained alternatively or in addition by applying to a mechanical stimulus to the matrix during the tissue growth, as will be described below.

The mechanical stability of the hybrid fascia material according to the invention can be enhanced by providing a completely resorbable support layer, in particular in the form of a mesh, on which the synthetic matrix is disposed. In another advantageous arrangement, the support layer can be arranged between two synthetic matrices of the invention. Resorbable, commercially available meshes can be used as support layers. If a support layer is used, the aforedescribed bio-mechanical requirements apply to the entire matrix structure.

According to another preferred embodiment of the invention, the surface of the polymer matrix can include a bio-active coating and/or composition. In particular, the polymer like structure. Especially for relatively long resorption times, the membrane or foil is advantageously also provided with pores that promote an exchange of nutrients and metabolic products. The synthetic matrix can have a rough or smooth surface. It has preferably a surface texture that promotes immobilization of tissue cells, in particular of fibrocytes.

According to a particularly preferred embodiment of the invention, the surface of the synthetic matrix is structured to induce oriented three-dimensional tissue growth. For this purpose, the surface structure is preferably straight or wavy, and/or has groove-shaped or point-shaped recesses on the matrix surface. For example, the structure can be applied to the matrix during or after its fabrication. In this way, an “anisotropic” fascia tissue similar to the natural fascia can be induced along a preferred direction. The same result can be obtained alternatively or in addition by applying to a mechanical stimulus to the matrix during the tissue growth, as will be described below.

The mechanical stability of the hybrid fascia material according to the invention can be enhanced by providing a completely resorbable support layer, in particular in the form of a mesh, on which the synthetic matrix is disposed. In another advantageous arrangement, the support layer can be arranged between two synthetic matrices of the invention. Resorbable, commercially available meshes can be used as support layers. If a support layer is used, the aforedescribed bio-mechanical requirements apply to the entire matrix structure.

According to another preferred embodiment of the invention, the surface of the polymer matrix can include a bio-active coating and/or composition. In particular, the polymer matrix can be coated with special inhibitors before being populated with the tissue cells (e.g., fibrocytes), which inhibit enzymes, in particular metalloproteinase, that can break down the matrix. Suitable inhibitors for enzymes capable of breaking down the matrix have been reported by Rudek et al. (M. A. Rudek, L. Venitz, W. D. Figg: Matrix metalloproteinase inhibitors: do they have a place in anticancer therapy? Pharmacotherapy. 2002, 22 (6), 705-720). This can reduce undesirable degradation of the collagen tissue, i.e. the neofacia, freshly synthesized on the matrix.

The polymer used for the matrix should be physically-chemically inert, should preferably not cause reactions with foreign bodies and should not be carcinogenic.

Preferably, degradable polymers are used, such as bio-degradable, thermoplastic elastomers that represent a polymer system. A polymer system is a group of polymers where different macroscopic properties, for example mechanical properties or a breakdown time of the polymer, can be varied almost independently by making small changes in the chemical structure. Because these polymer systems can be easily changed, they are promising candidates for the next generation of resorbable implant materials. Preferably, the synthetic polymer includes a macrodiol composed of at least one monomer, in particular a block copolymer based on different macrodiols. These are fabricated by producing macrodiol components from suitable, in particular cyclical monomers, by (ring-opening) polymerization. Suitable monomers are, in particular, c-caprolactone, di-glycolide, dilactide, (L,L-lactide or rac-dilactide) and/orp-dioxanone, which have already been approved for medical purposes. Biocompatible and simultaneously bio-resorbable multi-block copolymers are produced based on these macrodiols matrix can be coated with special inhibitors before being populated with the tissue cells (e.g., fibrocytes), which inhibit enzymes, in particular metalloproteinase, that can break down the matrix. Suitable inhibitors for enzymes capable of breaking down the matrix have been reported by Rudek et al. (M. A. Rudek, L. Venitz, W. D. Figg: Matrix metalloproteinase inhibitors: do they have a place in anticancer therapy? Pharmacotherapy. 2002, 22 (6), 705-720). This can reduce undesirable degradation of the collagen tissue, i.e. the neofacia, freshly synthesized on the matrix.

The polymer used for the matrix should be physically-chemically inert, should preferably not cause reactions with foreign bodies and should not be carcinogenic.

Preferably, degradable polymers are used, such as bio-degradable, thermoplastic elastomers that represent a polymer system. A polymer system is a group of polymers where different macroscopic properties, for example mechanical properties or a breakdown time of the polymer, can be varied almost independently by making small changes in the chemical structure. Because these polymer systems can be easily changed, they are promising candidates for the next generation of resorbable implant materials. Preferably, the synthetic polymer includes a macrodiol composed of at least one monomer, in particular a block copolymer based on different macrodiols. These are fabricated by producing macrodiol components from suitable, in particular cyclical monomers, by (ring-opening) polymerization. Suitable monomers are, in particular, ε-caprolactone, di-glycolide, dilactide, (L,L-lactide or rac-dilactide) and/or p-dioxanone, which have already been approved for medical purposes. Biocompatible and simultaneously bio-resorbable multi-block copolymers are produced based on these macrodiols by co-polymerization. The thermoplastic elastomers are elastic at room temperature (RT) and have at RT a high ultimate elongation ε_{R} of between 650 and 1100% at elasticity modules between 34 and 90 MPa. On one hand, these are preferably multi-block copolymers with crystallizable solid segments of poly (p-dioxanone) and an amorphous segment, for example poly[(L-lactide)-co-glycolide] with a glycolide fraction of approximately 15 mole %. Suitable are also multi-block copolymers that include a crystallizable poly-(ε-caprolactone) segment in addition to the hard poly-p-dioxanone segment. The thermoplastic elastomers are produced by co-condensation of two or more different macrodiol components with a bifunctional linking unit (for example, di-isocyanate, diacid dichloride, or phosgene). For attaining the desired mechanical properties, large molecular weights M_{W} of at least 100,000 g/mole are preferred. Molecular parameters of these polymer systems are molecular weight, microstructure (sequence), and co-monomer ratio of the macrodiols and the hard segment fraction in the multi-block copolymer. Hydrolytic breakdown tests of different co-polyester urethanes in buffer solution with pH 7 at the 37° C. and 70° C. have shown that the new materials can be completely broken down hydrolytically in vitro within one year or less, whereby the breakdown rate can be varied over a wide range. These polymers and their properties are described in A. Lendlein & R. Langer (Biodegradable, elastic shape-memory polymers for potential biomedical applications. Science 2002, 296 (5573), 1673-1676). These tested thermoplastic elastomers, unlike many polyhydroxy-carbonyl-acids, begin to lose weight very early, which continues nearly linearly during the entire breakdown process.

In addition to the thermoplastic elastomers, a number of semi-crystalline or completely amorphous, covalent cross-linked polymer network systems can be used as a polymer material for the synthetic matrix. In particular, different poly(ε-caprolactone)dimethacrylate with molecular weights in a range between 1,500 and 10,000 g/mole were synthesized. These cross-linked materials can be produced without the addition of a photo-initiator simply through irradiation with UV light. The poly(ε-caprolactone) network exhibits melting temperatures between 32 and 52° C. None of the tested samples had a glass transition in the temperature range between −20 and 70° C., where the networks are elastic. The mechanical properties of the materials were investigated at RT and at 70° C. The elasticity module at RT is in the range of 2 to 72 MPa. The tensile strength σₘₐₓ reaches values between 0.4 to 16.2 MPa, while the ultimate elongation ε_{R} varies between 16 and 290%. At 70° C., the curves of the tensile force vs. elongation are typical for elastomers. The elasticity modules at 70° C. are smaller by two orders of magnitude than at RT. Hydrolytic breakdown tests of different poly (ε-caprolactone) networks in buffer solution with pH 7 at 37° C. and 70° C. have shown that they lose less than 5% weight during one year in vitro. The breakdown time could be accelerated by introducing up to 15 mole % of diglycolide as a co-monomer, with the weight loss at the end of one year reaching 50% in vitro. Defined hydroxyl-functionalized oligomers were produced as precursors for the fabrication of amorphous polymer networks by ring-opening copolymerization of rac-dilactides and diglycolide and from the hydro-functionalized initiators in the form of di-, tri-, and tetrafunctional initiators. The end groups of these oligomers were cross-linked using an aliphatic diisocyanate as cross-linking agent. In DSC experiments, these networks show only a glass transition and no melting point, i.e., the polymer networks are amorphous and hence transparent. Above the glass transition temperature, which is in a range of 45° C. to 65° C., the materials exhibit rubber-like elastic properties. The elasticity module hereby decreases by a factor of 200 to 600 MPa. The elasticity module at 25° C. is between 300 and 600 MPa at tensile strengths omax between 30 and 45 MPa and ultimate elongations ε_{R} of 250 to 430%. The desorbability was determined in buffer solution with pH 7 at 37° C. and varies with the diglycolate contents, from almost hydrolytically inert to a weight loss of 90% within 100 days at a diglycolate contents of 50 mole %.

In addition to the aforedescribed polymers, other known biocompatible polymers, in particular biodegradable polymers, can be used as a synthetic matrix.

Hybrid Matrix System

The implantable hybrid matrix system of the invention includes essentially the aforedescribed matrix structure populated with individual tissue cells and/or regionally contiguous tissue structures and/or a completely formed neofascia. Tissue structure and neofascia are characterized by the presence of extra-cellular matrix (glycokalix), collagen, and elastin. The synthetic matrix can be populated with cells on one side or on both sides.

The tissue cells are preferably fibrocytes, fibroblasts, myoblasts, immobile connective tissue cells, stem cells, or mixtures thereof. The cells originate preferably from the fascia transversalis, the fascia lata, or other fasciae, or from the skin or of the ligament motion apparatus. Preferably, the cells are fibrocytes of the fascia transversalis. The cells can be autologous or allogenic or xenologic cells, preferably autologous cells. By combining the biocompatible matrix material with autologous cells, i.e., cells harvested from the patient, a structure with excellent tissue compatibility is obtained which results in the formation of an autologous fascia that immunologically matches the fascia of the patient.

Method for Generating/Inducing a Neofascia

Induction of a neofascia on the matrix structure can in principle be achieved in three different ways. According to a first variant, the tissue cells are immobilized on the polymeric matrix structure outside the body and subsequently cultivated in vitro, whereby depending on the duration of the cultivation, a more less confluent cell carpet, individual tissue growth sites or even an intact tissue (neofascia) are obtained. Only then is the produced hybrid matrix system implanted in the patient. According to the second variant, the cells are also deposited on the matrix structure outside the body, but are implanted immediately thereafter without prior cultivation. According to the third variant, the matrix structure of the invention is implanted as such, without being populated, so that the cells are immobilized only in vivo. According to the second and third variant, the tissue is entirely formed inside the body. Conversely, with the first variant, the tissue can be formed initially in vitro or in vivo, with tissue formation continuing inside the body.

Both primary and conditioned cells can be used to populate the polymeric matrix according to the first and second variant of the method. To prolong the limited transfer time of the tissue cells, in particular of human fibrocytes, the cells can advantageously be immortalized by inducing an ectopic expression of the reverse transcriptase subunit of human telomerase (hTERT). This enhances the proliferation of the cell culture and hence also the growth and survival time. In particular, fibrocytes of the fascia transversalis can initially be harvested surgically (by a minimally invasive procedure) from the hernia patient. These primary autologous cells can then be conditioned in culture and immortalized, whereafter they are immobilized in vitro on the polymeric matrix. The obtained Tissue Engineering product is transplanted after an additional cultivation, during which formation of a neofascia is at least initiated.

With all three variants of this method, a directed cell growth is desired for producing a tissue with biomechanical properties that approach as closely as possible those of the tissue to be repaired. The first method described above uses for this purpose a matrix with a suitable surface structure, which may already be provided, for example, by a corresponding network structure of the matrix or by pores and/or grooves on the mesh, foil or membrane. According to another method for inducing directed tissue growth, a mechanical stimulus is applied to the polymeric matrix structure alternatively or in addition. This stimulus can be applied, in particular, by alternatingly stretching the matrix structure in one direction and then relaxing the matrix structure. For this purpose, the matrix is clamped and, for example, stretched and relaxed by applying a motor force.

1. Matrix structure for induction of a neofascia having at least one two-dimensional matrix of at least one biocompatible synthetic polymer, which can be broken down in vivo within a resorption time, wherein the matrix has a surface that is suitable to immobilize tissue cells and to induce formation of tissue on the surface in vitro and/or in vivo.

2. Matrix structure according to claim 1, characterized in that the matrix has selected mechanical properties, in particular a mechanical load-carrying capacity that match at least approximately the mechanical properties of the tissue to be generated.

3. Matrix structure according to claim 2, characterized in that the matrix has a tensile strength of at least 35 N, in particular at least 40 N, preferably at least 50 N.

4. Matrix structure according to claim 2, characterized in that the matrix has an elasticity of at least 2.5 N/cm, in particular at least 3.0 N/cm, preferably at least 4.0 N/cm.

5. Matrix structure according to claim 1, characterized in that the resorption time of the polymer in vivo is selected so that a change in the mechanical properties of the matrix due to resorption is at least approximately compensated by the tissue forming on the matrix.

6. Matrix structure according to claim 1, characterized in that the resorption time of the polymer in vivo is selected so that the matrix breaks down completely only when tissue capable of carrying a mechanical load is formed.

7. Matrix structure according to claim 1, characterized in that the resorption time is at most 12 months, in particular between 3 and 6 months.

8. Matrix structure according to claim 1, characterized in that the matrix is a membrane or a foil or has a mesh-type structure.

9. Matrix structure according to claim 1, characterized in that the matrix is capable of inducing oriented three-dimensional tissue growth on its surface.

10. Matrix structure according to claim 9, characterized in that the matrix has a surface structure that induces the oriented cell growth, that the surface of the matrix includes in particular straight or wavy, groove-like recesses or point-shaped recesses.

11. Matrix structure according to claim 1, characterized in that the matrix comprises pores.

12. Matrix structure according to claim 1, characterized in that the matrix is disposed on a resorbable support layer, in particular a support mesh.

13. Matrix structure according to claim 12, characterized in that the support layer is arranged between two matrices.

14. Matrix structure according to claim 1, characterized in that the matrix comprises a bio-active coating and/or composition.

15. Matrix structure according to claim 14, characterized in that the matrix is treated, in particular coated, with natural or synthetic inhibitors for enzymes that can breakdown the matrix, and/or with growth factors.

16. Matrix structure according to claim 1, characterized in that the synthetic polymer is a thermoplastic elastomer, in particular a polymer system thereof.

17. Matrix structure according to claim 16, characterized in that the synthetic polymer comprises a macro-diol made of at least one monomer, in particular a block-copolymer based on different macro-diols.

18. Matrix structure according to claim 17, characterized in that the at least one monomer is selected from the group consisting of ε-caprolactone, di-glycolide, dilactide, (L,L-lactide or rac-dilactide) and p-dioxanone.

19. Matrix structure according to claim 1, characterized in that the synthetic polymer comprises a semi-crystalline or completely amorphous, covalently crosslinked polymer network system.

20. Matrix structure according to claim 19, characterized in that the semi-crystalline or completely amorphous polymer network system is based on poly(ε-caprolactone)dimethylacrylate, in particular a photo-crosslinked network of poly α-caprolactone) dimethyl.

21. Hybrid matrix system for inducing a neofascia, comprising a matrix structure according to claim 1, as well as tissue cells immobilized on the matrix surface.

22. Hybrid matrix system according to claim 21, characterized in that the immobilized tissue cells comprise fibrocytes, fibroblasts, myoblasts, immobile connective tissue cells, stem cells, or mixtures thereof.

23. Hybrid matrix system according to claim 21, characterized in that the tissue cells are cells of the fascia transversalis, the fascia lata, or of other fasciae, or cells of the skin or of the ligament motion apparatus.

24. Hybrid matrix system according to claim 21, characterized in that the tissue cells are autologous or allogenic or xenologic cells, in particular autologous cells.

25. Hybrid matrix system according to claim 21, characterized in that the tissue cells exist on the matrix individually, or form a cell carpet and/or form at least partially connected tissue structures.

26. Hybrid matrix system according to claim 21, characterized in that the matrix comprises the immobilized tissue cells and/or tissue structures on one side or on both sides.

27. (canceled)

28. Method for generating a neofascia, wherein a matrix structure according to claim 1 with at least one matrix made of at least one resorbable, synthetic, biocompatible polymer is used, and wherein a surface of the matrix is at least partially populated with tissue cells in vitro and/or in vivo, and wherein growth of the immobilized cells is induced on the matrix surface to form the neofascia.

29. Method according to claim 28, characterized in that the tissue cells are initially immobilized on the matrix in vitro, are cultivated in vitro, in particular until a cell carpet or at least partially connected tissue structures or an intact neofascia are formed, and the populated matrix is implanted.

30. Method according to claim 28, characterized in that the tissue cells are initially immobilized on the matrix in vitro, and that growth of the cells on the matrix is induced after implantation of the populated matrix to form a neofascia.

31. Method according to claim 28, characterized in that the tissue cells are immobilized in vivo, and that the tissue growth is induced in vivo after implantation of the unpopulated matrix structure.

32. Method according to claim 28, characterized in that oriented tissue growth of the matrix in a preferred direction is induced in vitro and/or in vivo by using a matrix structure with a structured surface.

33. Method according to claim 28, characterized in that oriented tissue growth of the matrix in a preferred direction is induced in vitro by applying a mechanical stimulus to the matrix structure, in particular by alternatingly stretching and relaxing the matrix structure.

34. Method according to claim 28, characterized in that tissue cells are used that are applied in the form of primary or conditioned and optionally immortalized cells.

35. Method according to claim 28, characterized in that the tissue cells comprise fibrocytes, fibroblasts, myoblasts, immobile connective tissue cells, stem cells, or mixtures thereof.

36. Method according to claim 28, characterized in that the tissue cells are cells of the fascia transversalis, the fascia lata, or of other fasciae, or cells of the skin or of the ligament motion apparatus.

37. (canceled)

38. (canceled)

39. Method for treatment of hernias, in particular of hiatal or inguinal hernias, wherein primary or conditioned tissue cells from a patient are immobilized, optionally cultivated, on a matrix structure according to claim 1, and wherein the matrix with the cells immobilized thereon is implanted.
